(19) United States
(12) Patent Application Publication
(10) Pub. No.: US 2026/0198880 A1
(43) Pub. Date: Jul. 16, 2026

(54) MULTI-VIEW DENTAL IMAGING SENSOR HOLDER

(71) Applicant: Shayda Cullen, Holden, MA (US)

(72) Inventor: Shayda Cullen, Holden, MA (US)

(21) Appl. No.: 19/379,180

(22) Filed: Nov. 4, 2025

Related U.S. Application Data
(63) Continuation-in-part of application No. 19/016,757, filed on Jan. 10, 2025, now Pat. No. 12,458,312.

(51) Int. Cl. A61B 6/51; A61B 6/00; A61B 6/58

(52) U.S. Cl. CPC A61B 6/512; A61B 6/4452; A61B 6/587

(57) ABSTRACT

A dental imaging sensor holder is disclosed that securely supports a dental imaging sensor while enabling multiple mounting configurations. In one embodiment, the holder includes opposing arms or grasping portions that receive and retain the sensor, the arms being adjustable to accommodate different sensor sizes. Connector channels or pivoting extensions formed on the holder allow attachment of a holder arm in different orientations, eliminating the need for multiple dedicated holders. The holder arm supports a bite block and an aiming ring coupler so that an aiming ring can be aligned with the sensor regardless of configuration. In certain embodiments, the holder further includes detent mechanisms, gear-and-tooth engagements, or rotatable bosses that permit indexed positioning of the sensor relative to the holder arm. The system provides a versatile and reconfigurable structure for accurately aligning a dental imaging sensor with an imaging emitter while reducing the number of separate holders required.

### CROSS REFERENCE

This application is a continuation in part of U.S. application Ser. No. 19/016,757, filed Jan. 10, 2025, and issued as U.S. Pat. No. 12,458,312 on Nov. 4, 2025, the entirety of which is hereby incorporated by reference.

### FIELD OF THE INVENTION

The present invention relates generally to dental radiograph sensor holder system, and, more particularly, relates to a multi-configurable dental radiograph sensor holder system that can be reconfigured for the various views taken for dental radiography.

### BACKGROUND OF THE INVENTION

Dental radiography is used to allow dentists and other professionals see the condition of teeth, bones, and tissue in the mouths of patients. This allows, for example, dentists to identify cavities, pre-existing dental work, the state of bone supporting the teeth, and so on. Taking radiographs of teeth is often an unpleasant experience for the patient, and involves a system of rods, holders, and rings to position the film or sensor in the patient's mouth at the proper orientation to capture the desired view. Typically, a set of radiographs includes anterior views, posterior views, and bitewing views. Upper and lower views are commonly taken for each of these categories. For each view, the dental professional must select the appropriate arm, hanger coupling to couple the arm to the aiming ring, and sensor holder, then assemble these together, and place the film/sensor in the patient's mouth at the correct orientation, then aim the beam source at the film/sensor. Since, in a conventional sensor holder system there can be three different arms or handles, multiple sensor holders, and multiple hanger couplings, producing a set of radiograph views can take some time as the dental professional has to re-select, and then re-assemble components for each view. Furthermore, all of these attachments must be sterilized before being used with the next patient. This is not desirable for either the dental professional or the patient.

Therefore, a need exists to overcome the problems with the prior art as discussed above.

### SUMMARY OF THE INVENTION

In accordance with some embodiments of the inventive disclosure, there is provided a dental imaging sensor holder that incudes first and second opposing arms that each have a channel configured to receive an edge of a dental imaging sensor on opposing sides of the dental imaging sensor. There is a base connecting the arms, at least one arm being slidably movable relative to the base to accommodate sensors of different widths. And there is at least one connector channel formed on an exterior of at least one of the arms. The connector channel is configured to receive a holder arm in at least two different orientations.

In accordance with a further feature, each connector channel includes at least one retention shoulder extending partially across the channel to retain the holder arm within the channel.

In accordance with a further feature, the base includes a detent mechanism configured to selectively hold the first and second opposing arms at predetermined separations corresponding to widths of standard dental imaging sensors.

In accordance with a further feature, the connector channel extends through a corner region of one of the arms such that the holder arm can be inserted in either of two perpendicular directions.

In accordance with a further feature, the connector channel is square in cross-section and the holder arm is a rod having a square cross-section configured to slidably fit within the connector channel.

In accordance with a further feature, there is further included a plurality of connector channels arranged on different sides of the arms, each connector channel being shaped and sized to interchangeably receive the holder arm.

In accordance with a further feature, the first and second opposing arms each include an interior sensor-receiving channel configured to receive opposite edge portions of the dental imaging sensor.

In accordance with a further feature, the base is rigidly connected to one of the arms and slidably connected to the other arm to permit relative translation between the arms.

In accordance with a further feature, the base includes a guide rail and the slidable arm includes a mating groove that engages the guide rail to constrain motion of the slidable arm.

In accordance with a further feature, the connector channel is open to an exterior surface of the arm to allow insertion of the holder arm laterally into the connector channel.

In accordance with some embodiments of the inventive disclosure, there is provided a dental imaging sensor holder that includes a pair of opposing grasping portions configured to clasp opposing sides of a dental imaging sensor, a connector extension pivotably coupled to the grasping portions, the connector extension including an attachment bore, a holder arm having an attachment post insertable into the attachment bore in a first orientation or a second orientation perpendicular to the first orientation. The attachment post and the attachment bore each have a square cross-section to prevent relative rotation of the holder arm and the connector extension.

In accordance with a further feature, the connector extension includes both an axial bore and a cross bore, and the attachment post is selectively insertable into either the axial bore or the cross bore.

In accordance with a further feature, the connector extension is rotatable relative to the grasping portions between a plurality of indexed positions.

In accordance with a further feature, the connector extension and the grasping portions include detent features configured to retain the connector extension in at least one of the indexed positions.

In accordance with a further feature, the grasping portions are movable relative to one another to accommodate dental imaging sensors of different sizes.

In accordance with a further feature, the grasping portions include a gear-and-tooth engagement mechanism that constrains separation of the grasping portions.

In accordance with a further feature, there is further included a biasing member urging the grasping portions toward one another.

In accordance with a further feature, the holder arm is a rod having a square cross-section, and the attachment bore of the connector extension is a square bore sized to slidably receive the holder arm.

In accordance with a further feature, there is further included an aiming ring coupler mounted on the holder arm and configured to hold an aiming ring in alignment with the dental imaging sensor.

In accordance with a further feature, the aiming ring coupler is repositionable between a central position and an offset position relative to the holder arm.

Although the invention is illustrated and described herein as embodied in a multi-configurable dental radiograph sensor holder system, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

“In the description of the embodiments of the present invention, unless otherwise specified, azimuth or positional relationships indicated by terms such as “up”, “down,” “left,” “right,” “inside,” “outside,” “front,” “back,” “head,” “tail” and so on, are azimuth or positional relationships based on the drawings, which are only to facilitate description of the embodiments of the present invention and simplify the description, but not to indicate or imply that the devices or components must have a specific azimuth, or be constructed or operated in the specific azimuth, which thus cannot be understood as a limitation to the embodiments of the present invention. Furthermore, terms such as “first,” “second,” “third,” and so on are only used for descriptive purposes and cannot be construed as indicating or implying relative importance.

In the description of the embodiments of the present invention, it should be noted that, unless otherwise clearly defined and limited, terms such as “installed,” “coupled,” “connected” should be broadly interpreted, for example, it may be fixedly connected, or may be detachably connected, or integrally connected; it may be mechanically connected, or may be electrically connected; it may be directly connected, or may be indirectly connected via an intermediate medium. As used herein, the terms “about” or “approximately” apply to all numeric values, whether or not explicitly indicated. These terms generally refer to a range of numbers that one of skill in the art would consider equivalent to the recited values (i.e., having the same function or result). In many instances, these terms may include numbers that are rounded to the nearest significant figure. In this document, the term “longitudinal” should be understood to mean in a direction corresponding to an elongated direction of the article or structure being referenced. Those skilled in the art can understand the specific meanings of the above-mentioned terms in the embodiments of the present invention according to the specific circumstances.

The phrase “extends from” indicates a structural relationship between two or more portions of an article. For example, if A extends from B, that means, first, that A and B are connected or joined to each other, and that A includes an end, side, point, surface, or other terminus, that is remote from B, and the major portion of A is between that terminus and the interface where A and B meet. The terminus may be free, or it may be connected to yet another structural element. For example, if A extends from B to C, or said differently, A extends between B and C, it means that B and C are remote from each other, and A connects to both B and C.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1A is a first exploded view of a multi-configurable dental radiograph sensor holder system, in accordance with some embodiments.

FIG. 1B is a second exploded view of a multi-configurable dental radiograph sensor holder system, in accordance with some embodiments.

FIG. 1C is an exploded view detail of the configurable linkage of a multi-configurable dental radiograph sensor holder system, in accordance with some embodiments.

FIG. 1D is an exploded view detail of the rotatable sensor coupling of a multi-configurable dental radiograph sensor holder system, in accordance with some embodiments.

FIG. 2A shows a perspective view of a multi-configurable dental radiograph sensor holder system in an anterior view configuration, in accordance with some embodiments.

FIG. 2B shows an aiming view of a multi-configurable dental radiograph sensor holder system in an anterior view configuration, in accordance with some embodiments.

FIG. 3A shows a perspective view of a multi-configurable dental radiograph sensor holder system in a bitewing view configuration, in accordance with some embodiments.

FIG. 3B shows an aiming view of a multi-configurable dental radiograph sensor holder system in a bitewing view configuration, in accordance with some embodiments.

FIG. 4A shows a perspective view of a multi-configurable dental radiograph sensor holder system in a posterior view configuration, in accordance with some embodiments.

FIG. 4B shows an aiming view of a multi-configurable dental radiograph sensor holder system in a posterior view configuration, in accordance with some embodiments.

FIG. 5A shows a bottom view of a positioning ring having a detent feature, in accordance with some embodiments.

FIG. 5B shows a perspective view of the positioning ring of FIG. 5A.

FIG. 6 shows a perspective exploded view of the positioning ring of FIG. 5A being used with a sensor holder having corresponding detent features, in accordance with some embodiments.

FIG. 7 shows a perspective exploded view of a sensor holder and sensor, in accordance with some embodiments.

FIG. 8A shows a perspective view of an aiming ring coupler, in accordance with some embodiments.

FIG. 8B shows a perspective view of the aiming ring coupler of FIG. 8A being used to couple to an aiming ring, in accordance with some embodiments.

FIG. 9A shows a perspective view of a bite block height extender, in accordance with some embodiments.

FIG. 9B shows a perspective view of the bite block height extender of FIG. 9A being used with a bite block, in accordance with some embodiments.

FIG. 9C shows another perspective view of a bite block having a bite block height extender coupled to the bite block, in accordance with some embodiments.

FIG. 10 shows a perspective view of a multi configurable dental radiograph sensor holder and aiming system, in accordance with some embodiments.

FIG. 11 shows a plan side view of a multi-configurable dental radiograph holder system, in accordance with some embodiments.

FIG. 12 shows a perspective view of a dental imaging sensor holder, in accordance with some embodiments.

FIG. 13 shows a side/edge elevational view of a dental imaging sensor holder, in accordance with some embodiments.

FIG. 14 shows a side elevational view of a dental imaging sensor holder, in accordance with some embodiments.

FIG. 15 shows a top plan view of a dental imaging sensor holder, in accordance with some embodiments.

FIG. 16 shows a bottom plan view of a dental imaging sensor holder, in accordance with some embodiments.

FIG. 17A shows side view of a dental imaging sensor holder in an expanded configuration, in accordance with some embodiments.

FIG. 17B shows side view of a dental imaging sensor holder in a retracted configuration, in accordance with some embodiments.

FIGS. 18A and 18B show sequential side views of a dental imaging sensor holder receiving a dental imaging sensor, in accordance with some embodiments.

FIG. 19 shows a dental imaging sensor holder mounted for use in a first configuration, in accordance with some embodiments.

FIG. 20 shows a dental imaging sensor holder mounted for use in a second configuration, in accordance with some embodiments.

FIG. 21 shows a perspective view of a sensor holder system in a bitewing configuration, in accordance with some embodiments.

FIG. 22 shows the sensor holder system of FIG. 21 looking through the aiming ring, in accordance with some embodiments.

FIG. 23 a perspective view of a sensor holder system in a lower configuration, in accordance with some embodiments.

FIG. 24 shows a detail of the interconnection of the holder bar and swivel arm of the dental imaging sensor holder system of FIG. 21 for the bitewing configuration, in accordance with some embodiments.

FIG. 25 shows a detail of the interconnection of the holder bar and swivel arm of the dental imaging sensor holder system of FIG. 21 for upper or lower view configuration, in accordance with some embodiments.

FIG. 26 shows a perspective view of a dental imaging sensor holder, in accordance with some embodiments.

FIG. 27 shows a perspective view of a dental imaging sensor holder of FIG. 26 with a cover removed to show the inside detail, in accordance with some embodiments.

FIG. 28 shows an exploded perspective view of a dental imaging sensor holder system, in accordance with some embodiments.

FIG. 29 shows a perspective view of a dental imaging sensor holder system in a first configuration.

FIG. 30 shows a perspective view of a dental imaging sensor holder system in a second configuration, in accordance with some embodiments.

FIG. 31 shows a perspective view of a dental imaging sensor holder system in a third configuration, in accordance with some embodiments.

FIG. 32 shows a cutaway detail of a distal end of a holder arm and a connector, in accordance with some embodiments.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The present invention provides a novel and efficient multi-configurable dental radiograph sensor holder system that allows reconfiguration of the system without having to disassemble and use different components to provide for different orientations of the sensor for the various dental views. in the following drawings, the inventive multi-configurable dental radiograph sensor holder system is shown in various exploded and assembled views, as well as detail views. Reference numerals are carried forward throughout the drawings to refer to the same structure across the drawings, and reference should be made to the multiple drawings through the following description.

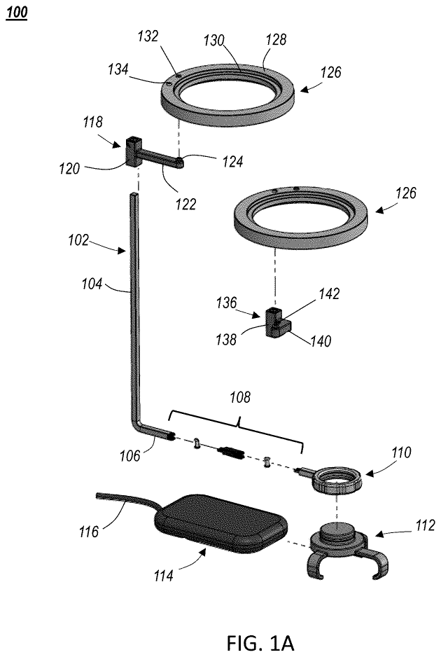

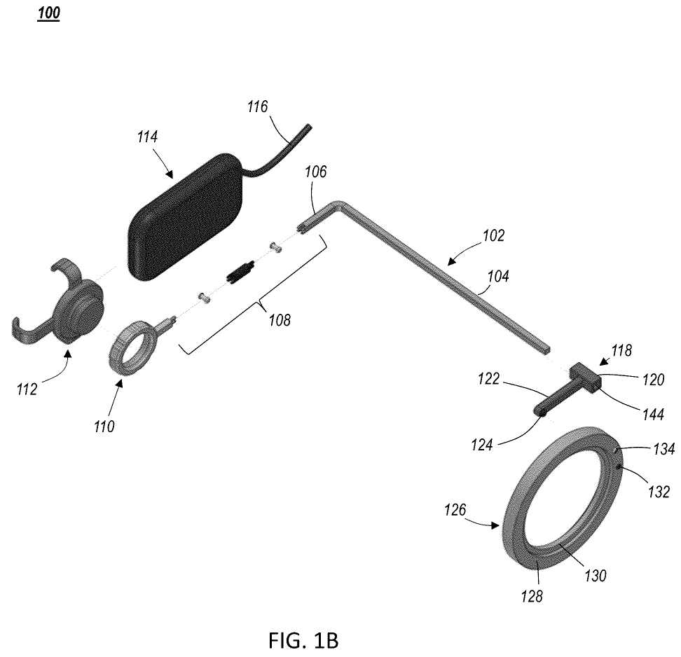

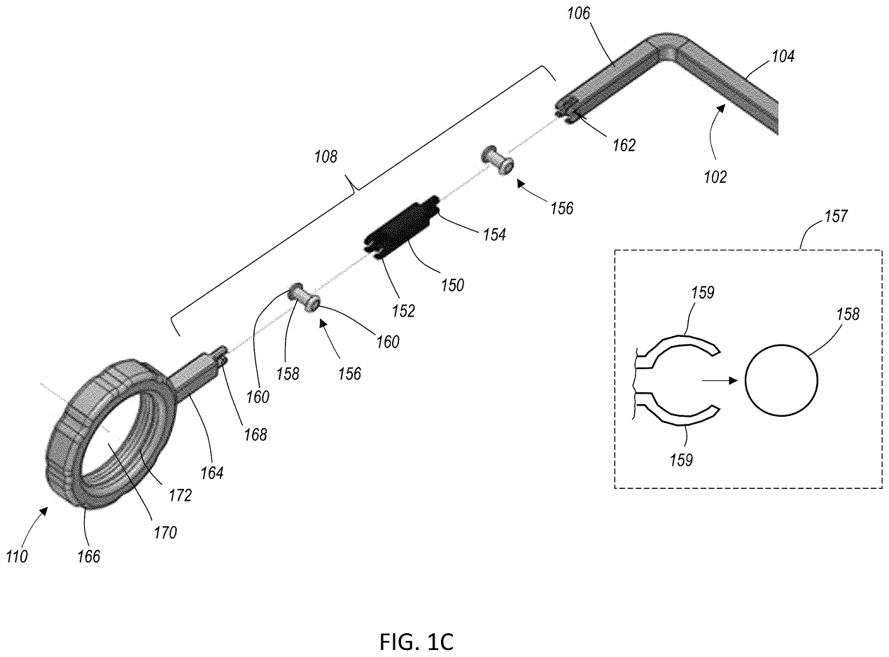

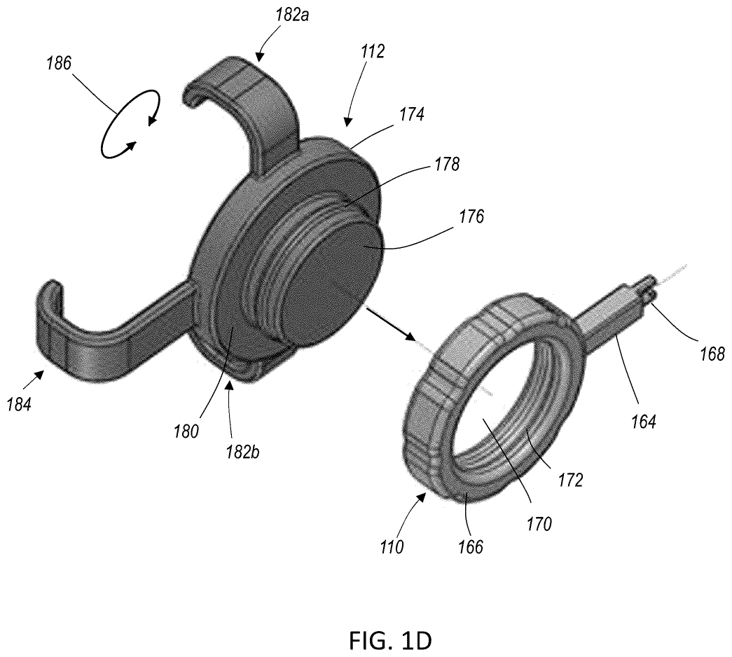

FIG. 1A is a first exploded view of a multi-configurable dental radiograph sensor holder system 100, in accordance with some embodiments. Similarly, FIG. 1B is a second exploded view of a multi-configurable dental radiograph sensor holder system. A handle 102 is formed by a square rod having a long portion 104 and a short portion 106. The long portion is generally oriented perpendicular to the plane of the sensor 114, and in the general direction of the beam emission that is emitted at the sensor 114. The short portion 106 is at a right angle to the long portion 106 and they meet at respective ends. The handle 102 is coupled to an aiming ring 126 by a hanger coupling 118 for bitewing views or hanger coupling 136 for anterior and posterior views. The hanger coupling 118, 136 includes body portion 120, 138, respectively, having a square bore 144 through it to allow the body portion 120, 138 to fit onto the long portion 104 of the handle 102, and move along the long portion 104 as needed. Each of the handle couplings 118, 136 have an extension arm 122, 140, respectively, that couples to the aiming ring 126 at either of several coupling location 132, 134. Specifically, hanger coupling 118 is used and couples to coupling location 132 for bitewing views, and hanger coupling 136 is used and coupled to coupling location 134 for anterior and posterior views. A coupling peg 124, 142, respectively, fit into the coupling locations 132, 134, which are openings through the body 128 of the aiming ring 126. The aiming ring 126 also includes an alignment ledge 130 which is a recessed relative to the body 128 to receive the distal end cylindrical beam source barrel, as is well known.

To couple the sensor 114 to the system in the correct alignment, a sensor holder 112 is provided that is rotatably coupled to a sensor positioning ring 110 which is coupled to the short leg 106 of the handle 102 through a hinge linkage 108. The sensor 114 is rectangular with rounded corners so as not to poke into the tissue in a patient's mouth, and fits into an arrangement of three hook arms 182a, 182b, 184 (see FIG. 1D) which fit tightly against the outside of the sensor 114. By making the sensor holder 112 rotatable with respect to the sensor positioning ring 110 the sensor can be rotated when switching from viewing the left side of the patient's mouth to the right side, or front, as desired, allowing the sensor cable 116 a direct path out of the patient's mouth. The hinge linkage 108 allows the sensor positioning ring 110 to be positioned in any of several positions by articulating the linkage 108 into the desired configuration/position.

FIG. 1C is an exploded view detail of the configurable hinge linkage 108 of a multi-configurable dental radiograph sensor holder system, in accordance with some embodiments. The linkage 108 includes an intermediate portion 150 that in general has a similar cross section shape to that of the handle 102. It is an elongated article with a square cross section. At the opposite ends 152, 154 of the intermediate portion 150 are clasps that are configured to snap over the cylindrical body 158 of hinge pins 156. The hinge pins also have caps 160 at each end of the cylindrical body 158 that extend outward, relative to the cylindrical axis of the cylindrical body 158 to retain the hinge pins 156 in the linkage system. Similarly, at the distal end 162 of short portion 106 of the handle 102 there is a clasp, and there is a clasp at the distal end 168 of the radial extension 164 of the sensor positioning ring 110. These clasps have opposing retaining fingers that snap over the cylindrical body 158 of a hinge pin 156, and the clasps nest together on the hinge pins 156. For example, the clasp at the end 168 of the radial extension 164 has one pair of retaining fingers, while the clasp at then end 152 of the intermediate portion 150 has two pairs of retaining fingers; the single pair of retaining fingers at end 168 fit between the two pairs of retaining finger at end 152 over the cylindrical body 158 of the hinge pin 156. The snap fit is achieved by making the retaining fingers arcuate, with their ends separated by a distance less than the diameter of the cylindrical body 158. By pushing the retaining fingers over the cylindrical body 158, the ends of the retaining fingers are displaced apart until they are on the other side of the cylindrical body 158 where they are urged back together by the spring property of the material. Thus, there is a hinge pin 156 disposed between the distal end 162 of the short portion 106 and a first end 156 of the intermediate portion 150 which creates a first hinge point. A second hinge point is created by a second hinge pin 156 being disposed between the second end 152 of the intermediate portion 150 and the distal end 168 of the radial extension 164 of the positioning ring 110. Both of these hinge points restrict articulation to the same plane in order to be configured for the various views. Inset 157 show a side profile the cylindrical body 158 of a hinge pin 156, which retaining fingers 159 being moved over the cylindrical body 158. The hinge linkage 108 thus provides at least two hinge points, which are at the locations of the hinge pins, about which the hinge linkage can articulate. Both of these hinge points allow articulation in the same plane, i.e. in a plane parallel to the plane of the aiming ring when the aiming ring is properly attached to the handle 104.

The sensor positioning ring 110 includes a ring body 166 that forms a circular opening 170 through the body 166. The radial extension 164 can extend along a radial line from the ring body 166. On the inner surface of the ring body 166 there can be annular ridge and grove retaining features 172 that capture, through interference, corresponding ridge feature(s) 178 on the outer surface of the cylindrical interface portion 176 of the sensor holder 112, as shown in FIG. 1D. The cylindrical portion 176 is a puck-shaped circular structure that extends from the main body 174 of the sensor holder 110. There is a shoulder 180 against which the body 166 of the sensor positioning ring 110 will be adjacent when the sensor holder 112 and the sensor positioning ring 110 are assembled together. This is done by pushing the cylindrical portion 176 into the opening 170 so that an annular ridge around the outer side surface of the cylindrical portion 176 is captured in an annular groove 172 around the inner surface of the body 166 of the sensor positioning ring 110. This allows the sensor holder 112 to rotate while coupled to the sensor positioning ring 110, as indicated by arrow 186. Opposing hook arms 182a and 182b hold the sensor 114 on opposing sides of the sensor 114, and end hook arm 184 provides both a stop and additional support for holding the sensor 114.

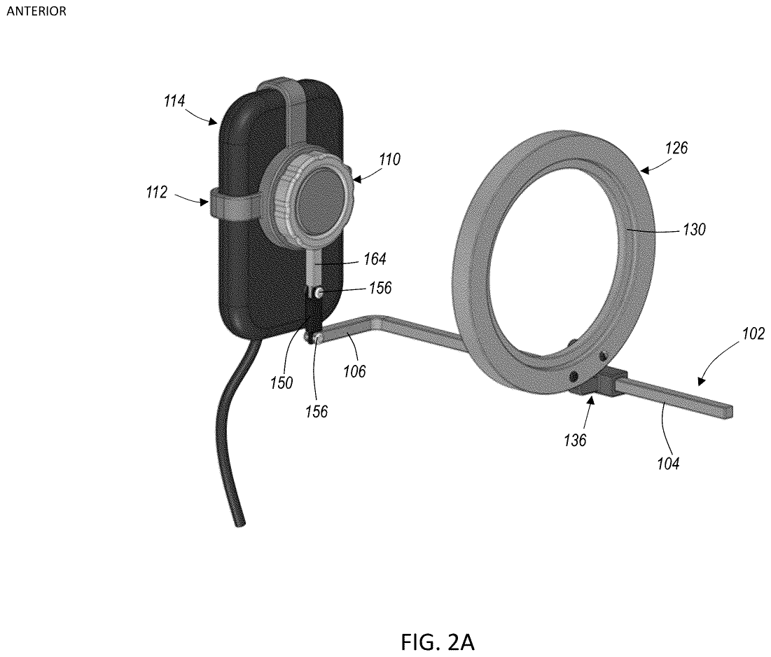

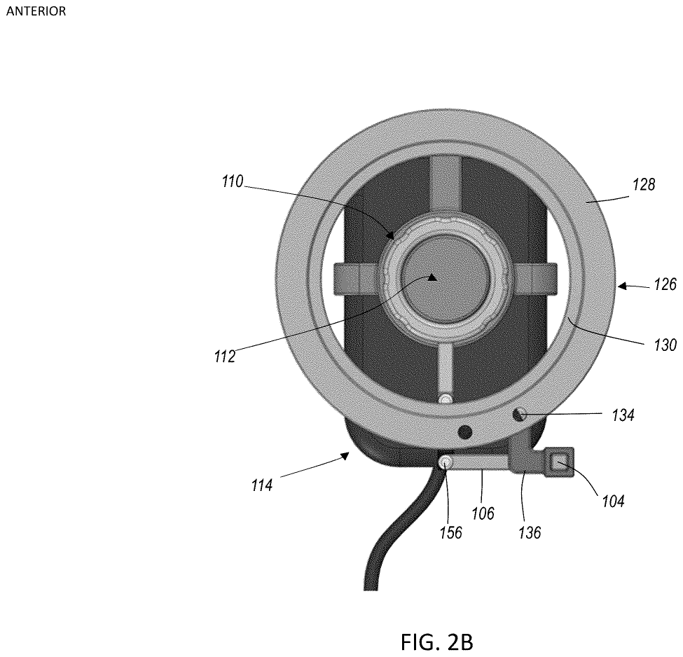

In general, to assembly the system 100, the dental professional selects either the bitewing handle coupling 118 or the anterior/posterior handle coupling 136 for bitewing views or anterior/posterior views, respectively. Then the body portion 120, 136 of the selected handle coupling 118, 136 is placed over the long portion 104 of the handle 102. The hinge linkage 108 can be pre-assembled with the sensor positioning ring 110. The sensor 114 an be placed into the sensor holder 112, which is snapped into the sensor positioning ring 110 as described above. The selected handle coupling 118, 136 is further coupled to the aiming ring 126. Then the hinge linkage can be adjusted to the desired position for the desired radiograph view. FIGS. 2A-2B, 3A-3B, and 4A-4B show various configurations of the system 100 for anterior, bitewing, and posterior views, respectively.

FIG. 2A shows a perspective view of a multi-configurable dental radiograph sensor holder system in an anterior view configuration, and FIG. 2B shows an aiming view of a multi-configurable dental radiograph sensor holder system in an anterior view configuration, in accordance with some embodiments. In this configuration, the intermediate portion 150 of the hinge linkage is oriented at right angle to the elongated direction of the short portion 106 of the handle 102, and is co-linear with the radial extension 164 of the sensor positioning holder. The sensor 114 is oriented/rotated so that its elongated direction is oriented vertically. To produce the view, the end of the emitter barrel is placed into the aiming ring 126 and briefly energized to emit a beam toward the sensor 114, which produces an image of the teeth that are between the sensor 114 and the aiming ring 126/emitter. The position of the aiming ring 126 can be adjusted along the handle 102 by sliding the handle coupling 136 to the desired position. As shown, the system 100 is configured to produce an image of the upper anterior teeth, and can be flipped over to produce and image of the mandibular anterior teeth. The coupling pin of the handle coupling 136 fits into coupling opening 134 so that the aiming ring 126 is centrally positioned with respect the sensor 110 in this configuration.

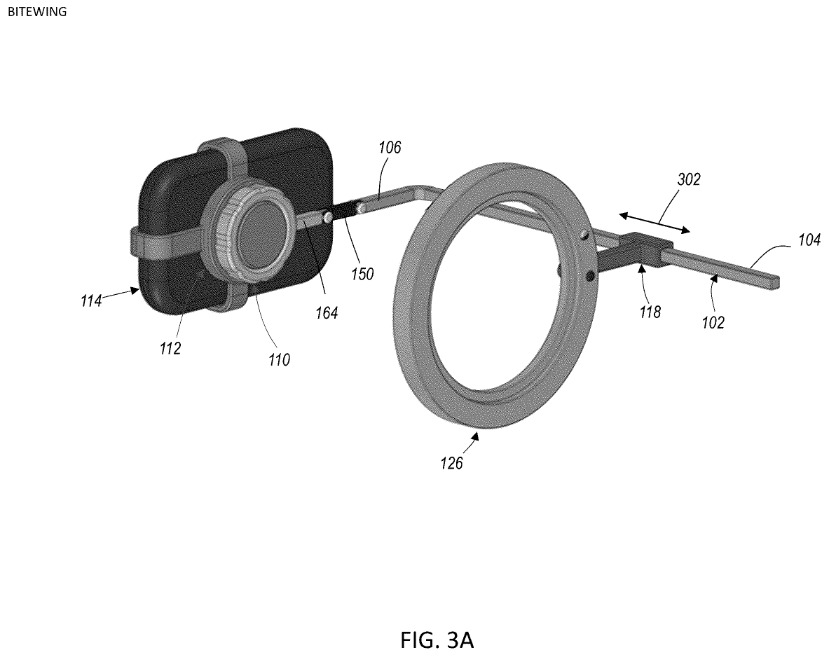

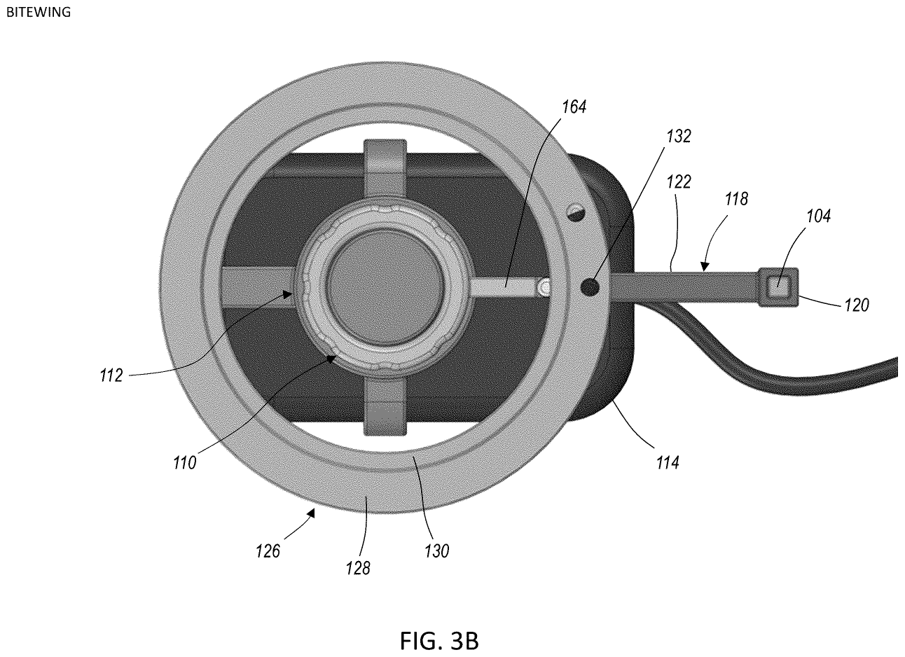

FIG. 3A shows a perspective view of a multi-configurable dental radiograph sensor holder system in a bitewing view configuration, and FIG. 3B shows an aiming view of a multi-configurable dental radiograph sensor holder system in a bitewing view configuration, in accordance with some embodiments. In the bitewing view configuration, the hinge linkage is entirely co-linear; the short portion 106 of the handle 102, intermediate portion 150, and radial portion 164 are all aligned in line with each other. The sensor 114 is oriented/rotated so that the elongated direction of the sensor 114 is generally horizontal. The bitewing handle coupling 118 is used to couple the handle 102 to the aiming ring 126, and the coupling peg 124 is placed into coupling opening 132 to centrally align the aiming ring 126 with the sensor 114.

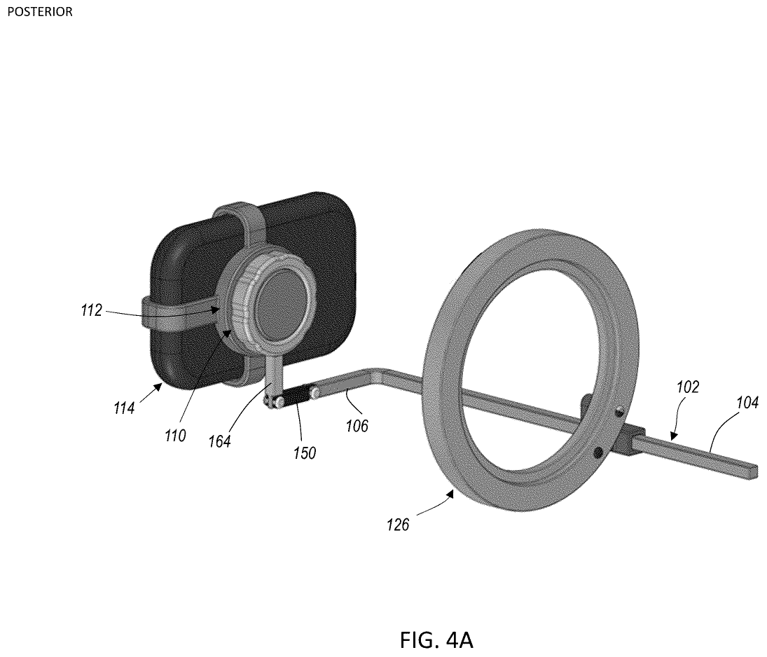

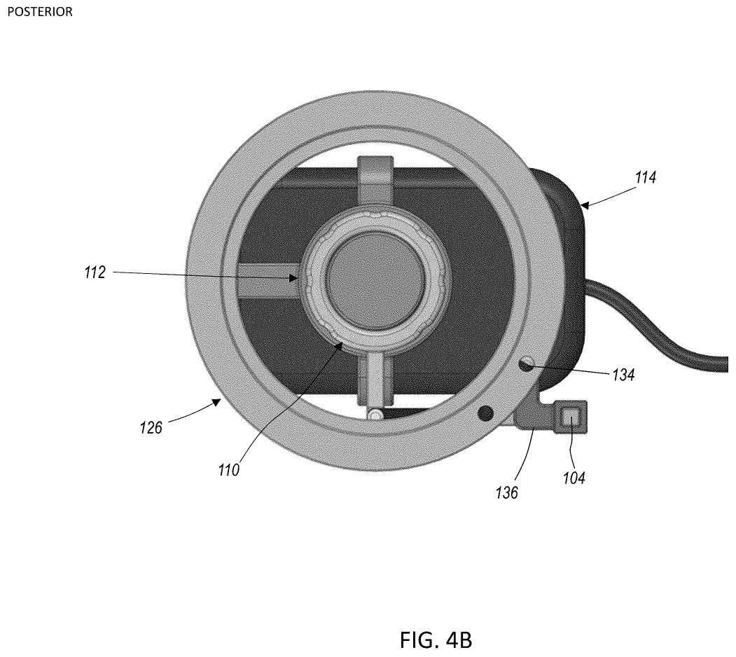

FIG. 4A shows a perspective view of a multi-configurable dental radiograph sensor holder system in a posterior view configuration, and FIG. 4B shows an aiming view of a multi-configurable dental radiograph sensor holder system in a posterior view configuration, in accordance with some embodiments. Again, here the handle coupling 136 is used, and coupled to coupling opening 134 in the aiming ring 126. The hinge linkage is configured by aligning the intermediate portion 150 to be co-linear with the short portion 106 of the handle 102. However, the radial extension 164 is turned at a right angle to the intermediate portion 150. The sensor 114 is oriented so that the elongated direction of the sensor 114 is generally horizontal.

In general, the aiming ring is circular in a plane that is perpendicular to the long portion of the handle. The hinge linkage is articulable within a plane that is parallel to the plane of the aiming ring, and the sensor holder is rotatable in a plane that is parallel to the plane of the aiming ring. The sensor holder also holds the sensor so that the sensor is oriented in a plane that is parallel to the plane of the aiming ring.

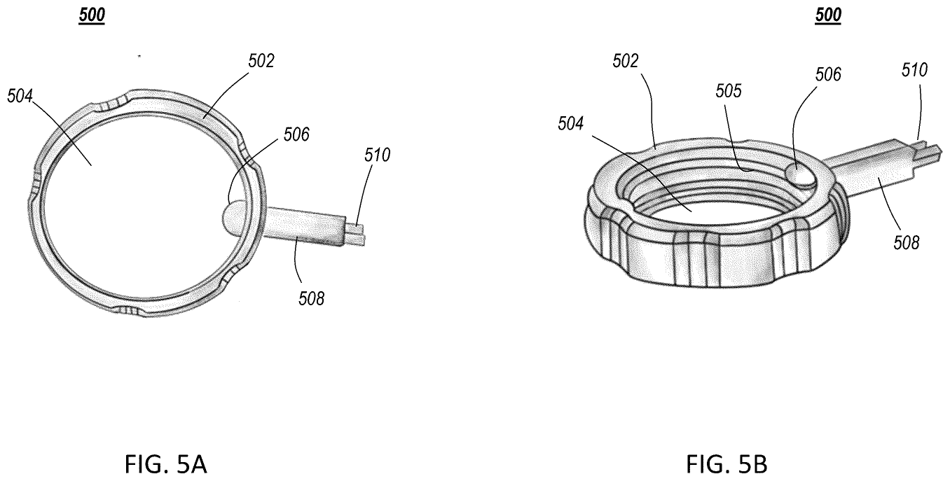

FIG. 5A shows a bottom view of a positioning ring 500 having a detent feature, in accordance with some embodiments, and FIG. 5B shows a perspective view of the positioning ring 500. The positioning ring 500 includes a circular body 502 through which there is a circular opening 504 that passes through the circular body 502. There is a positive or convex detent feature 506 that protrudes into the opening 504 from an inner wall 505. The positive detent feature can be either static (immovable) or active. If the detent feature 506 is active, it is biased outward from the circular wall, such as by a spring, into the opening 504. The positioning ring 500 includes a radial extension 508 that extends from the outside of the circular body 502, and includes a clasping feature 510 at the distal end of the radial extension 508 to connect the positioning ring to hinge linkage (e.g. 108).

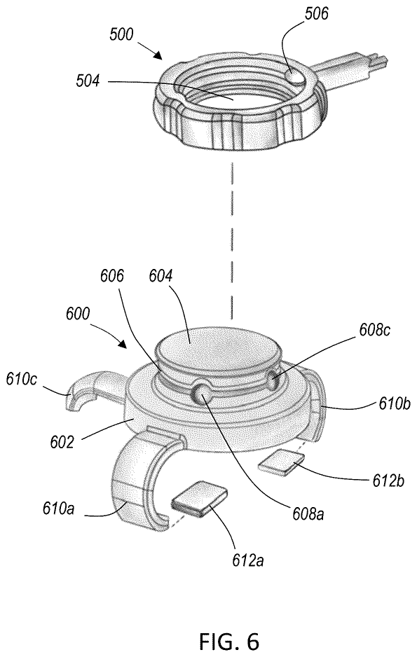

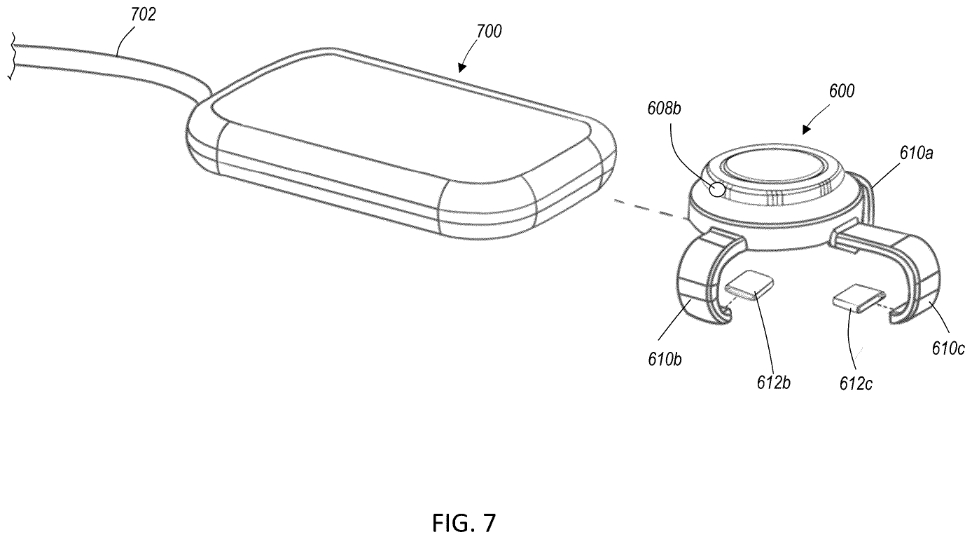

FIG. 6 shows a perspective exploded view of the positioning ring 500 being used with a sensor holder 600 that has corresponding detent features, in accordance with some embodiments. The sensor holder 600 includes a main body 602, and a cylindrical portion 604 that extends from the main body 602. The main body 602 is also cylindrical, and puck shaped, where the diameter of the main body is much larger than its height. The cylindrical portion 604 and the main body can share a common cylindrical axis (which is perpendicular to the radius of the cylindrical portion). The cylindrical portion 604 has a diameter that is sized to fit snugly into the opening 504 of the sensor positioning ring 500, and includes a sidewall 606 that has concave detent features 608a, 608b (see FIG. 7), and 608c. These concave detent features 608 are sized to create a mechanical interference with the detent feature 506 of the sensor positioning ring 500, which is convex. More specifically, when the sensor holder 600 is rotated to a position where the convex detent feature 506 meets one of the concave detent features 608a, 608b, or 608c, a mechanical interference between the convex detent feature 506 and the concave detent feature 608a, 608b, or 608c creates resistance to rotation of the sensor holder 600. This resistance can be overcome with a rotational force being manually applied to the sensor holder 600 relative to the positioning ring 500 that is more than the force needed to rotate the sensor holder 600 when the mechanical interference is not present (e.g. convex detent feature 506 is not in any of the concave detent features 608a-c). That is, the size of the cylindrical portion 604 is such that it can fit into the opening 504, and past the convex detent feature 506, and then rotate. When rotated, the detent feature 506 can fall into one of the concave detent features 608, which creates a mechanical interference that resists further rotation. This resistance be overcome with a small amount of rotational force applied to the sensor holder to allow the user to rotate the sensor holder to the desired position. These detent features 506, 608, define several positions of the sensor holder 600 to the sensor positioning ring 500 that are useful for holding the sensor, to prevent the sensor holder from coming out of position when used for producing a radiograph image. These positions include a position for when the system is configured in an anterior view configuration, a position for a posterior view configuration, and a position for a bite wing view configuration, as exemplified in FIGS. 2A-4B. The senor holder also includes three hook arms 610a, 610b, 610c, which each extend from the main body 602 outward, and then down (in the opposite direction from the cylindrical portion 604), and then inward, in the form of a hook. The precise shape of these hook arms 610 is intended to follow the outer surface contour of a radiograph sensor body, so as to hold the radiograph sensor body snugly between the hook arms 610, where hook arm 610c is positioned to meet an end of the sensor body and hook arms 610a, b hold the sensor body at the sides.

To accommodate various sensor body sizes, flexible ends 612a, 612b, 612c (see FIG. 7 also) can be attached to the ends of the respective hook arms 610a-c. The flexible ends 612 can be sized to hold a specific sensor body size, and there can be multiple different sizes of the flexible ends that the user can select in accordance with the particular sensor being used. The flexible ends 612 can slip onto the distal ends of the hook arms 610, and can be made of a flexible material that creates more friction against the outer surface of the sensor body that the material of the sensor holder 600. In some embodiments, the flexible ends can be made of silicone, and can be sterilized in an autoclave, along with the sensor holder 600, positioning ring 500, and hinge linkage and other portions of the sensor holder and aiming structure. FIG. 7 shows a perspective exploded view of a sensor holder 600 and sensor 700, in accordance with some embodiments. The sensor holder 600 holds the sensor 700, as shown, for example, in FIGS. 2A-4B. The sensor 700 includes a housing, seen here, with either film or an electronic imaging detector inside the housing. In most cases, presently, film is no longer used and the sensor provides image data via a cable 702 to a computing system that can display the resulting image.

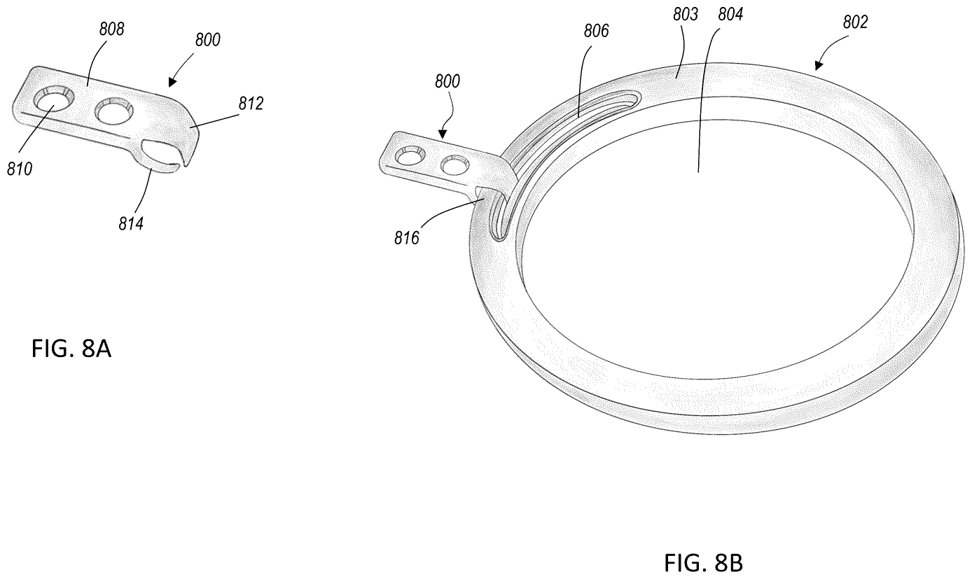

FIG. 8A shows a perspective view of an aiming ring coupler 800, in accordance with some embodiments, and FIG. 8B shows a perspective view of the aiming ring coupler 800 of FIG. 8A being used to couple to an aiming ring 802. The coupler 800 includes a flat body portion 808 that can have one or more openings 810 to attach the coupler 800 to a hanger coupling (e.g. 118) that can be moved along a handle (e.g. 102). The coupler 800 also includes opposing coupling portions 812, 814 that extend from the flat body portion 808 and one end. Portion 812 fits into an arcuate slot 806 in the circular body 803 of an aiming ring 802, while portion 814 traverses the outside of the aiming ring 802 and to the opposite side of the aiming ring 802 to grasp the outer section 816 of the body. The arcuate slot 806 extends along the body 803 in an arc that has a length selected to allow reconfiguration of the aiming ring to accommodate the various view configurations shown in FIGS. 2A-4B. Thus, the coupler 800 can be slid from one end of the arcuate slot 806 to the other, or to positions in between to provide finer adjustment of the positioning of the sensor relative to the aiming ring 802 than is there are only two positions available. That is, the aiming ring is mounted to the long portion of the handle by a coupler (e.g. 800) that fits into the arcuate slot 806, and wherein a position of the aiming ring 802 can be adjusted by rotating the aiming ring 802 such that the coupler moves to a selected position along the arcuate slot 806. This obviates the need to disconnect the aiming ring from the handle and swap hanger blocks having different length coupling arms, as in FIGS. 1A-4B.

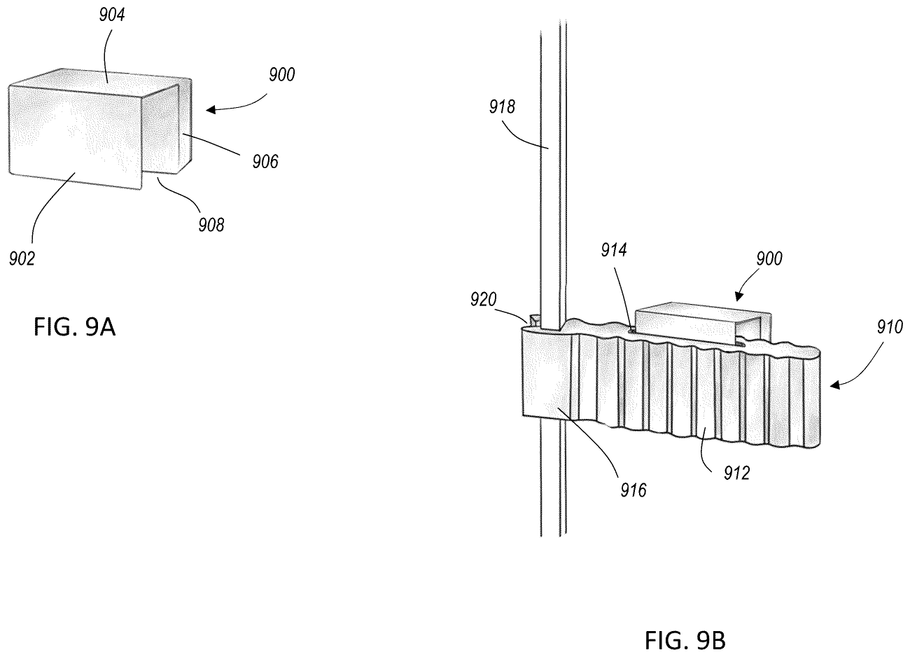

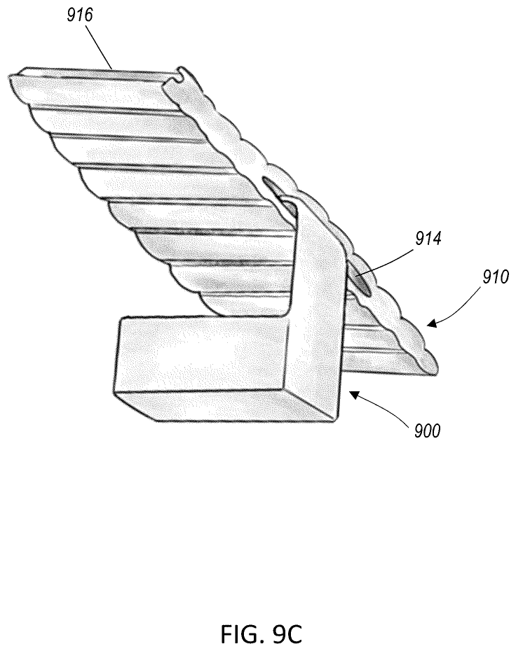

FIGS. 9A-9C shows perspective views of a bite block height extender 900 and bite block 910, in accordance with some embodiments. The bite block extender 900 includes an insert portion 902 that is remote from a main body 906. A wall 904 extends between the insert portion 902 and the main body 906, creating a space 908 between them, so as to allow the insert portion 902 to be inserted into a slot 914 in the bite block 910. The bite block 910 has a bite body 912 that patients bite down on when radiographic imaging is being performed to hold the sensor in position. The bite block includes an end portion 916 that is configured to clip onto a handle 918. The handle also supports the aiming ring and the sensor, as indicated in FIGS. 2A-4B. In particular, the end portion 916 can be formed into opposing extensions 920 that can be pushed over the handle 918 and hold onto the handle 918. The bite block 910 is made of a firm material, but does not need to be rigid so as to not be uncomfortable when placed in the mouth of a patient. As shown in FIGS. 9B-9C, the insert portion 902 of the bite block height extender 900 can be inserted into the side of the bite block 910 in slot 914. The distance between the insert portion 902 and the main body 906 allows the main body 906 to rest against the interior of the patient's oral cavity, and provide support in certain cases where use of the bite block 910 alone does not provide enough support. For example, in some cases the patient can have a short palate, or there can be an abscess in the patient's mouth that prevents use of the bite block 910 alone.

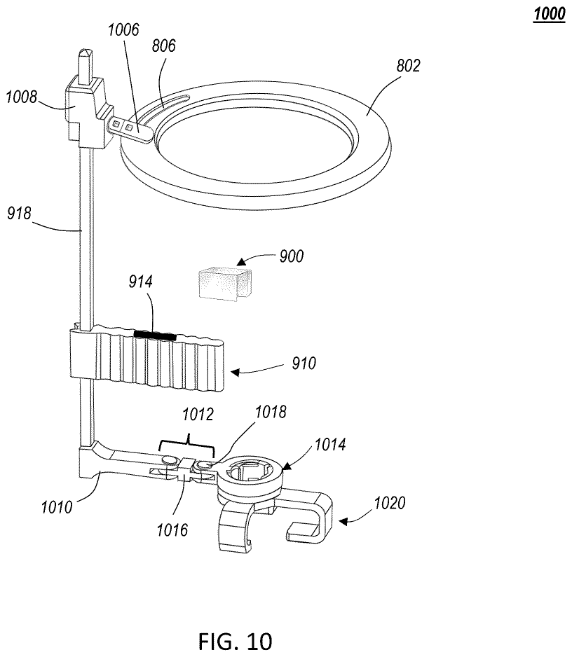

FIG. 10 shows a perspective view of a multi configurable dental radiograph sensor holder and aiming system 1000, in accordance with some embodiments. The system 1000 is intended to hold a radiograph imaging sensor in a patient's mouth/oral cavity in order to produce images of patent's teeth for examination by a dental professional, as shown, for example, in FIGS. 2A-4B. The system 1000 allows the positioning of the sensor to be reconfigured in order to produce images of various portions of the patent's dentition. In particular, posterior, anterior and bite wing images can be produced by reconfiguring the system, as indicted in FIGS. 2A-4B. That is, the system 1000 is a slight alternative to that shown in FIGS. 2A-4B to accomplish the same results and collect the same images as would be collected using the configurations of FIGS. 2A-4B. Accordingly, there is an aiming ring 802 that include an arcuate slot 806. The aiming ring is intended to allow alignment of a radiographic source that is incident on the sensor to produce the desired image, as is well known. The aiming ring is coupled rigidly to a handle 918 by a coupler 1006 and hanger coupling 1008. The coupler 1006 can couple to the aiming ring anywhere along the slot 806, and the hanger coupling 1008 can be moved along the shaft of the handle 918 as needed for a given patient. A bite block 910 is also coupled to the handle 918 to allow the patient to bite down on the bite block and hold the system 1000 in place to produce an image. In some cases, for some patients, a bite block height extender 900 may be used to provide additional support for the system 1000. The bite block 910 can also be moved along the shaft of the handle 918 to suit the particular dimension of a given patient's oral cavity. The handle can have a short portion 1010 that extends at a right angle, generally, giving the handle 918 an overall “L” shape. The short portion 1010 connects, though a hinge linkage 1012, to a sensor positioning ring 1014, which is in turn coupled to a sensor holder 1020. The hinge linkage 1012 includes an intermediate portion 1016 that is coupled to the short portion 1010 at one end, and the radial extension of the positioning ring 1014, via hinge pins 1018. Thus, the hinge linkage 1012 works in the same manner as hinge linkage 108 of FIG. 1C. Between the hinge linkage and the adjustable coupling to the aiming ring, the system 1000 can be configured to any of the configurations shown in FIGS. 2A-4B to collect anterior, posterior, and bite wing images.

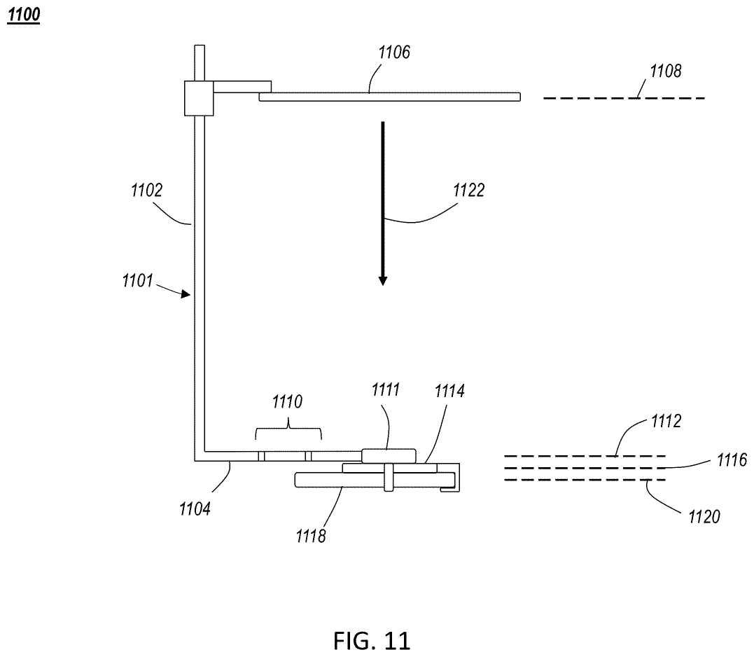

FIG. 11 shows a plan side view of a multi-configurable dental radiograph holder system 1100, in accordance with some embodiments. A handle includes a long portion 1102, and a short portion 1104 at right angles to each other. An aiming ring 1106 is mounted or coupled to the long portion 1102 of the handle 1101. The aiming ring is generally flat and circular within an aiming ring plane 1108. A hinge linkage 1110 couples the short portion 1104 of the handle to a positioning ring 1111, and the hinge linkage is articulable in a hinge plane 1112 that is parallel to the aiming ring plane 1108. The hinge linkage 1110 has at least two hinge points. In some embodiments the hinge linkage is designed to limit articulation to a single plane, so that the positioning ring 1111 can only be moved in one plane. A sensor holder 1114 is rotatably coupled to the positioning ring 1111, and is rotatable in a holder plane 1116, which is also parallel to the aiming ring plane 1108. This holds the sensor 1118 oriented in a sensor plane 1120 that is also parallel to the aiming ring plane. Thus, radiation is aimed through aiming ring, from a source, in the direction of arrow 1122, which is perpendicular to the aiming ring plane 1108, and the other planes 1112, 1116, 1120, and is incident on the sensor 1118 to produce an image. The sensor 1118 is positioned in the oral cavity of the patient, and the aiming ring 1106 is outside of the patient, adjacent the patient's face. Thus, by aligning the source perpendicular to the aiming ring plane, and centered on the aiming ring, the source will be properly aimed at the sensor, even though the user cannot see the senor in the patient's mouth.

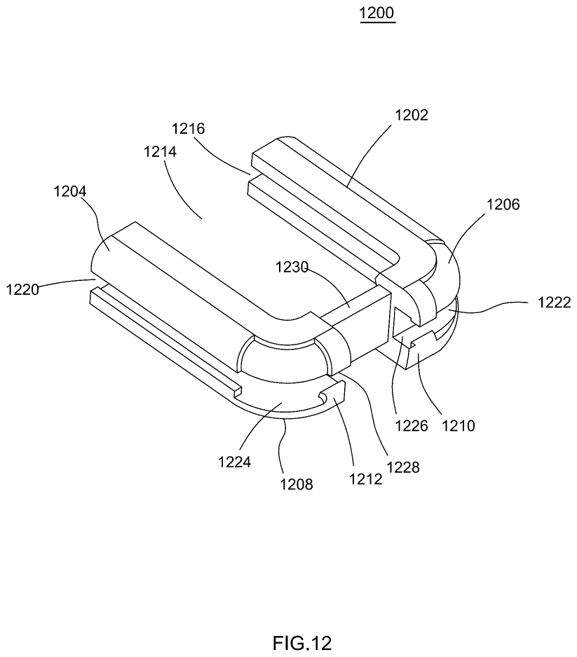

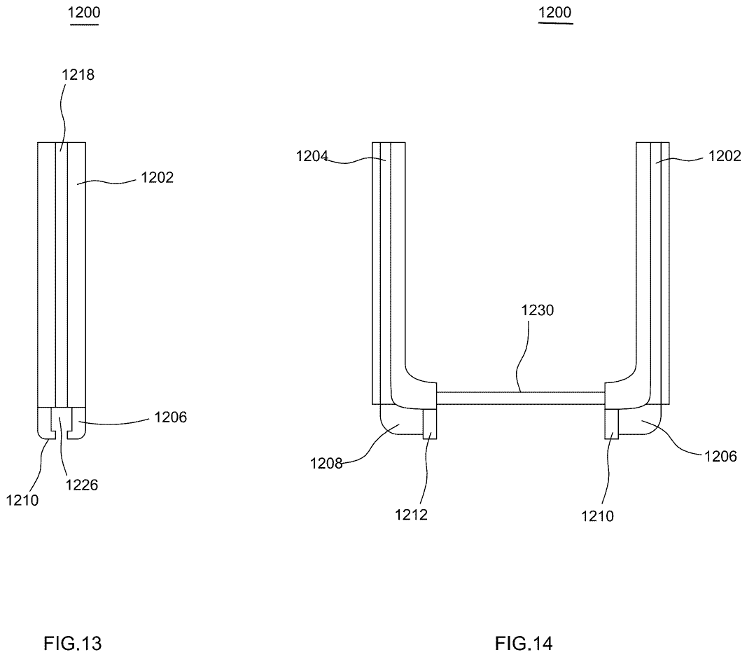

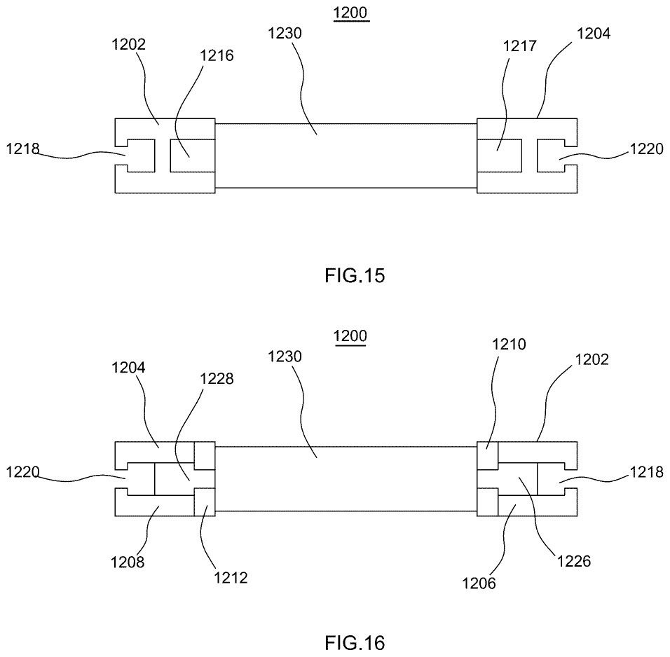

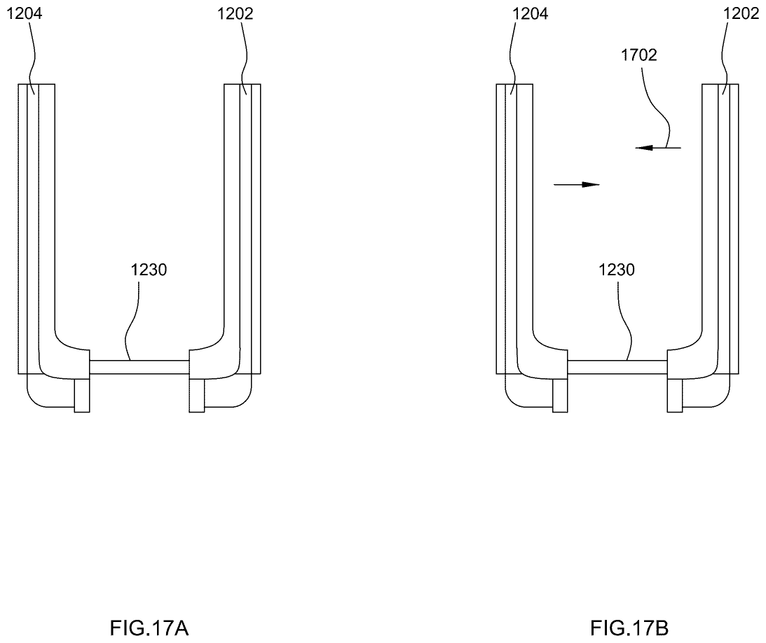

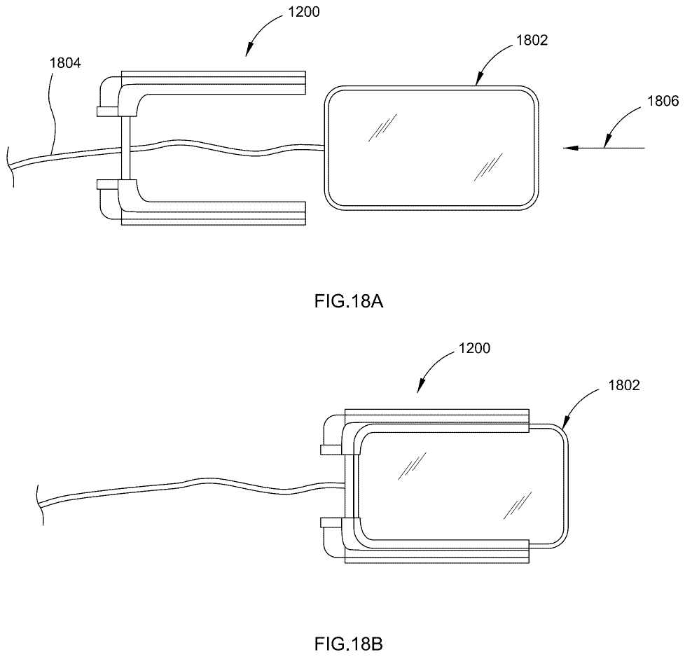

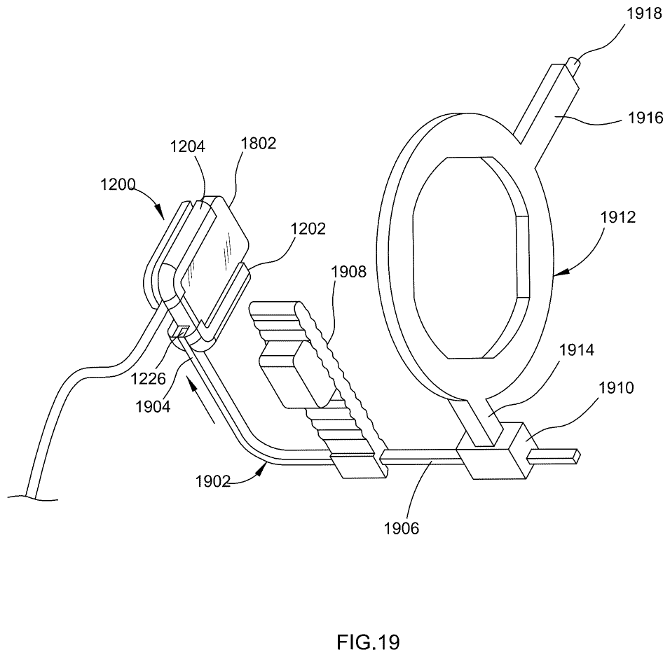

FIG. 12 shows a perspective view of a dental imaging sensor holder 1200, in accordance with some embodiments. Reference should further be made to FIGS. 13-16 as well in the following description, which show, respectively, a side/edge elevational view, a side elevational view, a top plan view, and a bottom plan view of the dental imaging sensor holder 1200. FIGS. 17A & 17B show an example of an operation of the sensor holder 1200. The sensor holder 1200 includes two opposing and parallel elongated portions or arms 1202, 1204. Each of these arms 1202, 1204 include an inner channel 1216, 1217 that is open to the space 1214 between the arms 1202, 1204. These channels 1216, 1217 are sized to receive and hold a dental imaging sensor. That is, the opposite side edges of a dental imaging sensor fit into the channels 1216, 1217, as is indicated, for example in FIGS. 18A & 18B, which show a dental imaging sensor 1802 being placed into the sensor holder 1200. In FIG. 18A the sensor 1802 is shown outside of the sensor holder 1200, with cable 1804 extending from the sensor 1802. Arrow 706 indicates a direction of movement of the sensor 1802 to progress from FIG. 18A to FIG. 18B in which the sensor 1802 is held by the sensor holder 1200 in the channels 1216, 1217 of the arms 1202, 1204. On the outside of the arms 1202, 1204, opposite from the space 1214 between the arms 1202, 1204, are channels 1218, 1220 that are square, and sized to receive an end portion of a holder arm. For example, in FIG. 19, holder arm 1902 has end portion 1904 that is an elongated member with a square cross section that can slide into either channel 1218, 1220.

Similarly, across the bottom of the holder 1200, are channels 1226, 1228 at the ends of the arms 1202, 1204. Channel 1228 in arm 1204 is perpendicular to channel 1220, and channel 1226 at the end of arm 1202 is perpendicular to channel 1218. Channels 1226, 1228 are likewise square and shaped/sized the same as channels 1218, 1220 to receive the end portion 1904 of holder arm 1902. Further, channels 1226, 1228 are axially aligned so that when the end portion 1904 passes through either channel 1226 or 1228, it will also pass through the other channel 1226, 1228 if pushed through. Channel 1218 and channel 1226 meet at opening 1222 at the corner 1206 of arm 1202, which allows the end portion 1904 to pass in either the direction of channel 1218 or channel 1226. Likewise, there is an opening 1224 in arm 1204 at corner 1208 where channel 1220 and channel 1228 meet, which allows insertion of the end portion 1904 through either channel 1220 or channel 1228. Each of the channels 1218, 1220, 1226, 1228 can be open along the outside of the holder. As shown here, each of the channels 1218, 1220, 1226, 1228 are open to the outside, and have opposing retention shoulders such as shoulder 1210, 1212, which extend inward and across a portion of the channel to partially close off the respective channel. This allows retention of the end portion 1904 of the holder arm 1902 in the channel, the prevents the end portion 1904 from coming out of a channel other than in an axial direction along the channel. Note that channels 1216, 1217 lack retention shoulders as the sensor 1802 fits into these channels 1216, 1217 and extends across space 1214 to the opposite channel 1216/1217.

A base 1230 connects the two arms 1202, 1204 together at their respective corners 1206, 1208. The base 1230 is a rigid member that extends between the two arms 1202, 1204, generally perpendicular to the elongated direction of the arms 1202, 1204, which is in the direction of channels 1216, 1217, 1218, 1220. However, at least one of the arms 1202, 1204 is slidably coupled to the base 1230, which allows the arms 1202, 1204 to move relative to each other, as indicated by arrows 1702. This allows different sized sensors to be held by the sensor holder 1200. The bridge 1230 can have detent features that hold the arms 1202, 1204 at known distances equal to the width of various known sensors.

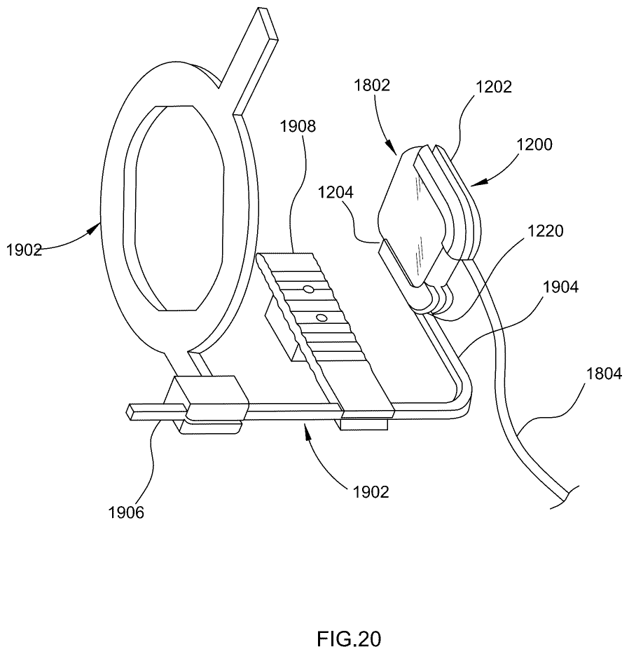

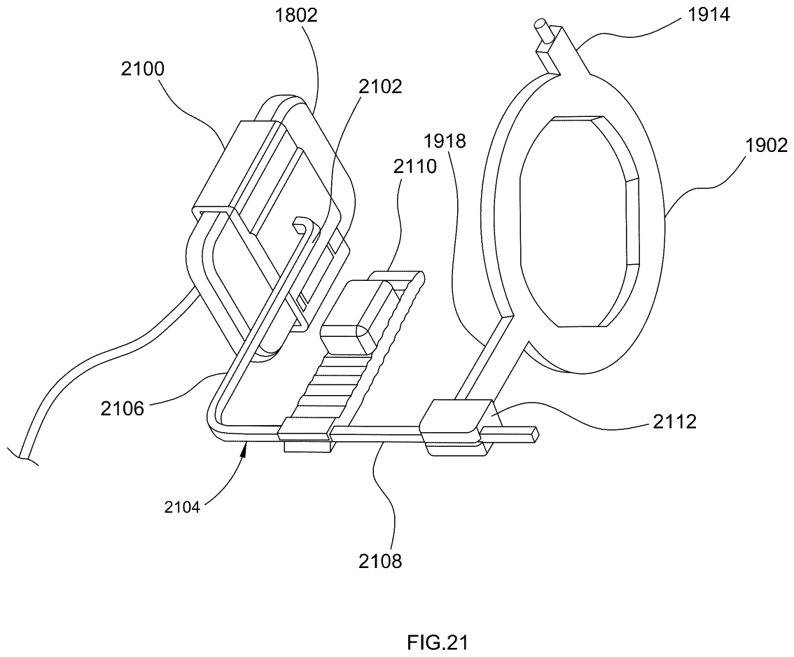

FIG. 19 shows a dental imaging sensor holder 1200 mounted for use in a first configuration, in accordance with some embodiments. The system includes a holder arm 1902 with can be a square rod that has an end portion 804 at a right angle to a main portion 1906. The end portion 1904 connects to the holder 1200 by being placed into one of the channels 1218, 1220 or 1226 & 1228. As shown here, the end of the end portion 1904 is passing through channel 1226 and can pass into channel 1228 as well. The sensor 1802 is seated in the space 1214 between arms 1202, 1204, and against the base 1230. On the main portion 1906 of the holder arm 1902 there is a bite block 1908 and an aiming ring coupler 1910. The aiming ring coupler allows an aiming ring 1912 to be attached to the coupler 1910 at either an offset leg 1914 or a central leg 1916, such as by inserting a protrusion 1918 into a corresponding opening into the coupler 1910. The choice of leg 1914, 1816 depends on the viewing configuration of the holder 1200 and sensor 1802 (e.g. for bitewing, anterior, or posterior view). FIG. 20 shows the dental imaging sensor holder 1200 mounted for use in a second configuration, with the end portion 1904 inserted into channel 1220 along leg 1204. Since the sensor 1802 is above the holder arm 1902, the aiming ring 1912 is coupled to the coupler 1910 with the offset leg 1914, as in FIG. 19. In use, with the system as shown in either of FIGS. 19 and 20, the sensor 1802 is placed into the mouth of a patient. The aiming ring will be outside of the patient's mouth and is aligned with the sensor 1802. That is, the plane of the aiming ring 1912 is parallel with the plane of the sensor 1802, and the opening of the aiming ring 1902 is generally centered over the sensor 1802. Thus, even though the sensor 1802 cannot be seen when placed into the patient's mouth, the head of the imaging emitter (not shown) only has to be aligned with the opening through the aiming ring 1912 to be aimed at the sensor 1802. Because the sensor holder 1200 can be connected to the holder arm 1902 in various positions, the need for multiple holders is eliminated. The same holder 1200 can be used for multiple different views, rather than needed separate components for each different view, which requires the operator to move the sensor to a different holder for each different view of the patient's teeth.

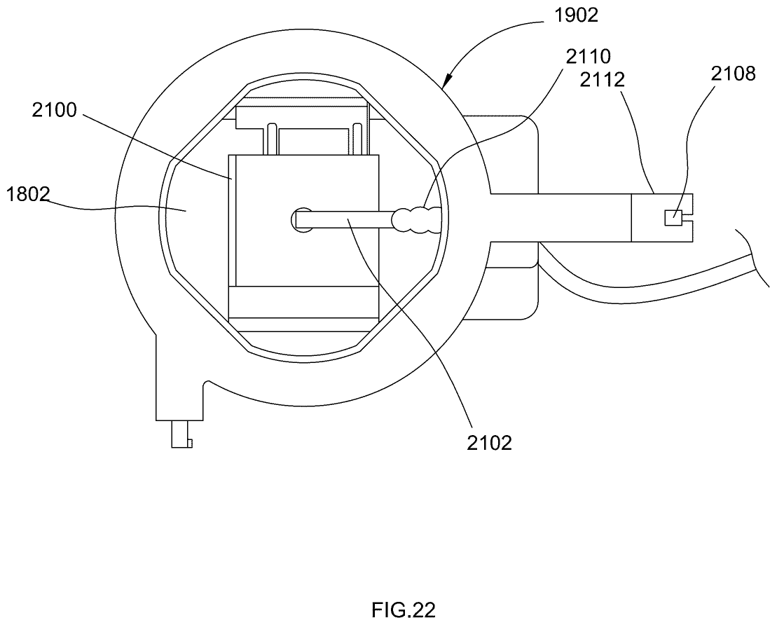

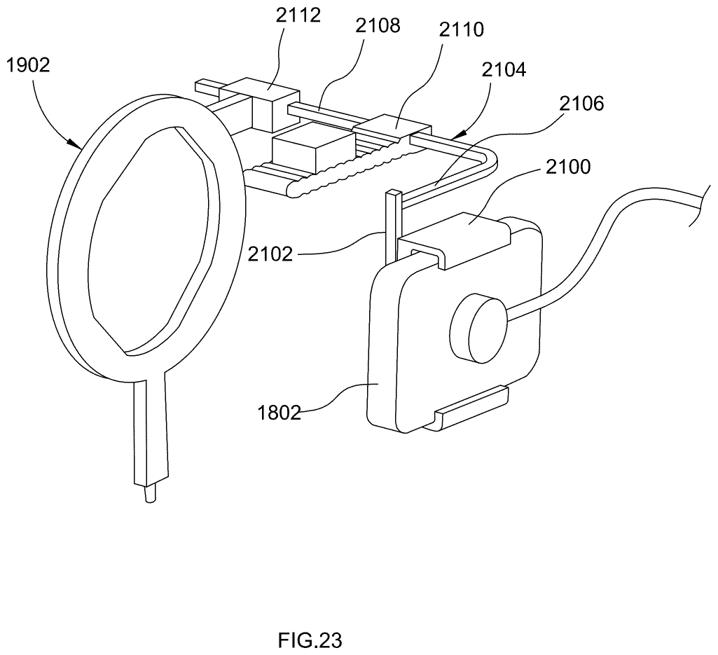

FIG. 21 shows a perspective view of a sensor holder system in a bitewing configuration, in accordance with some embodiments. In this system a different sensor holder 2100 is used which clasps the holder on opposing sides of the sensor 1802. The holder 2100 includes a pivoting connector extension 2102, which is an elongated bar position in a plane parallel to the plane of the sensor 1802 when the sensor 1802 is in the sensor holder 2100. The connector extension 2102 connects to the holder 2100 at a central point between the opposite sides of the holder 2100 and is configured to mate with the end portion 2106 of the holder arm 2104. The holder arm 2104 also has a main portion 2108 that is angle perpendicularly to the end portion 2104, and on which the bite block 2110 and the aiming ring coupler 2112 are located. The coupler 2112 can be identical to the coupler 1910 of FIG. 19. The aiming ring 1902 is shown here with the center leg 1918 attached to the coupler 2112. The holder arm 2104 is a square rod, meaning it is a rod having a square cross section and four flat sides. The coupler 2112 has a square channel through it, though which the main portion 2108 of the holder arm 2102 passes in a slight friction fit that holds the coupler 2112 in place, but allows an operator to move/slide the coupler 2112 along the length of the main portion 2108. As shown in FIG. 21, the system is configured for a bitewing view. In FIG. 22 the same system shown looking through the aiming ring 1902. In FIG. 23 the system shown in FIGS. 21 & 22 has been reconfigured for viewing the upper or lower teeth. Specifically, the connector extension 2102 is turned/pivoted ninety degrees relative to its position in FIG. 21, and the end potion 2106 of the holder arm 2104 mates with the connector portion 2102 at a ninety degree angle, rather than being coaxial as in FIG. 21. The aiming ring 1902 has also been repositioned so that the offset leg is connected to the coupler 2112 to position the aiming ring in correspondence with the position of the sensor 1802 relative to the holder arm 2104.

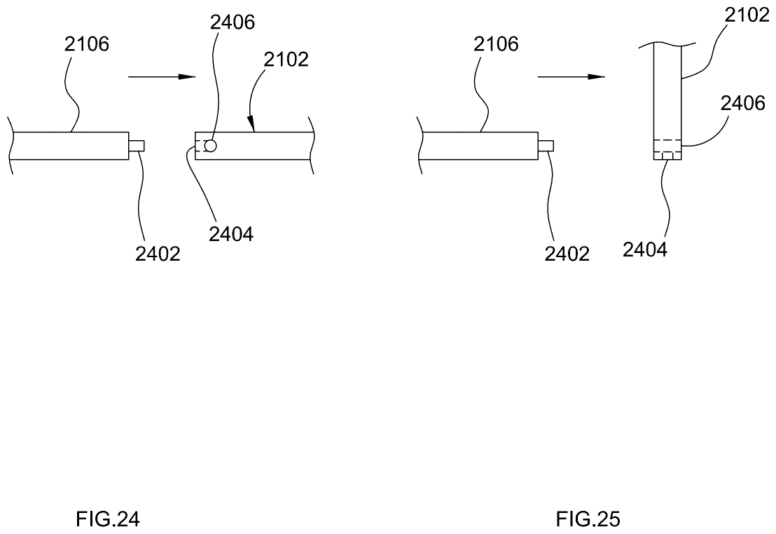

FIGS. 24 and 25 show details of the interconnection of the holder arm 2104 and the connector extension 2102 of the dental imaging sensor holder 2100 of the system of FIG. 21, for the bitewing configuration, and the offset configuration of FIG. 23, respectively, in accordance with some embodiments. The end portion 2106 of the holder arm 2104 includes an attachment post 2402 that extends from the end of the end portion 2106. The connector extension 2102 includes an axial bore 2404 into the connector extension 2102 along its axis, and a cross bore 2406 that passes through the connector extension in a direction perpendicular to the axis of the connector extension, the axis being defined along the elongated direction of the connector extension 2102. Thus, attachment post 2402 fits into either the axial bore 2404 (FIG. 24) or the cross bore 2406 (FIG. 25) in a friction fit, meaning that the attachment post 2402 is retained in the bore 2404, 2406 by friction. The attachment post 2402 and the bores 2404, 2406 can be square (having a square cross section) to prevent turning/twisting of the sensor holder 2100 relative to the holder arm 2104.

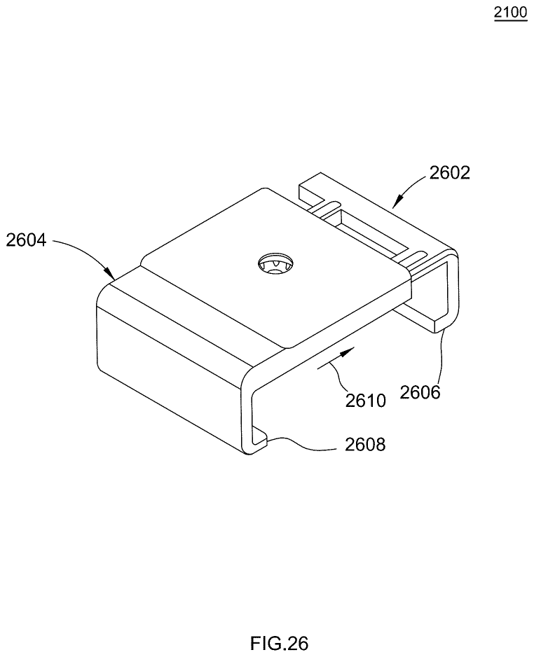

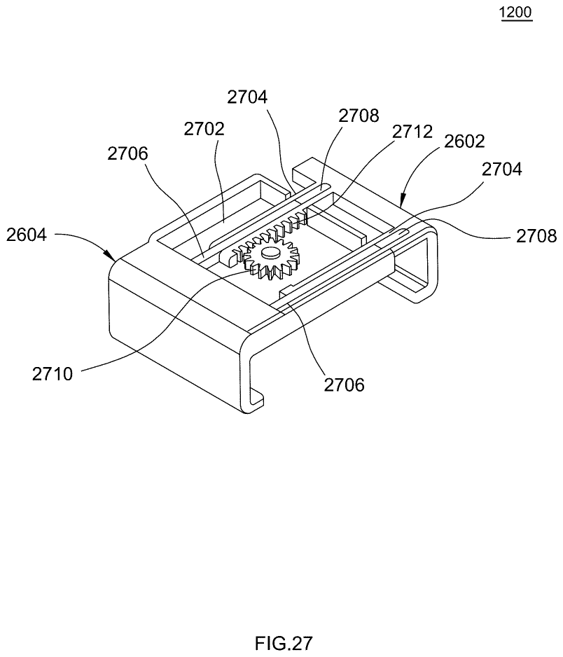

FIGS. 26 and 27 show perspective views of a dental imaging sensor holder 2100, with and with a cover portion, in accordance with some embodiments. The holder 2100 comprises a first grasping portion 2602 and a second, opposing grasping portion 2604. Each of the grasping portions 2602, 2604 have an end wall 2606, 2608, respectively, which extends around the outer side/edges of the sensor 1802 in an opposing arrangement to hold the sensor 1802. To allow for different sized sensors, the two grasping portions 2602, 2604 can be moved relative to each other as indicated by arrow 2610. In FIG. 27, where the cover portion is removed, it can be seen that there are legs 2704 that define a channel 2708 on grasping portion 2602 that sit on a surface of grasping portion 2604 such that a wall 2706 on the grasping portion 2604 slides along the channel 2708 in each instance. One side of the legs 2704 can have teeth 2712 that mesh with a gear 2710 that is pinned to grasping portion 2604. This can prevent the two grasping portion 2602, 2604 from coming apart by providing a large terminal tooth at the end of the teeth 2712 that is too big to mesh with the teeth of the gear 2710. In some embodiments a spring (not shown) can be used to draw the two grasping portion 2602, 2604 together and provide sufficient force to hold the sensor between the grasping portions 2602, 2604.

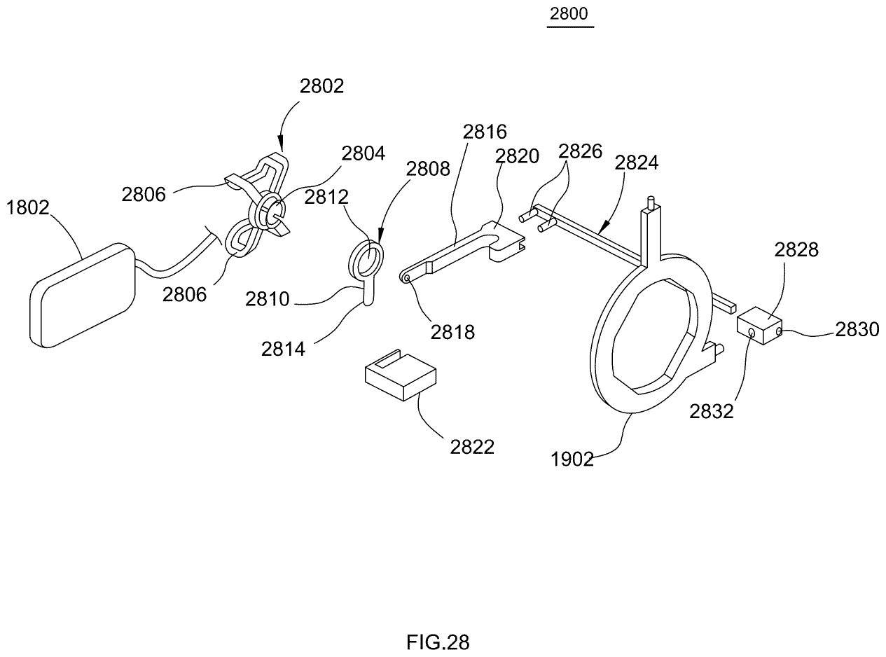

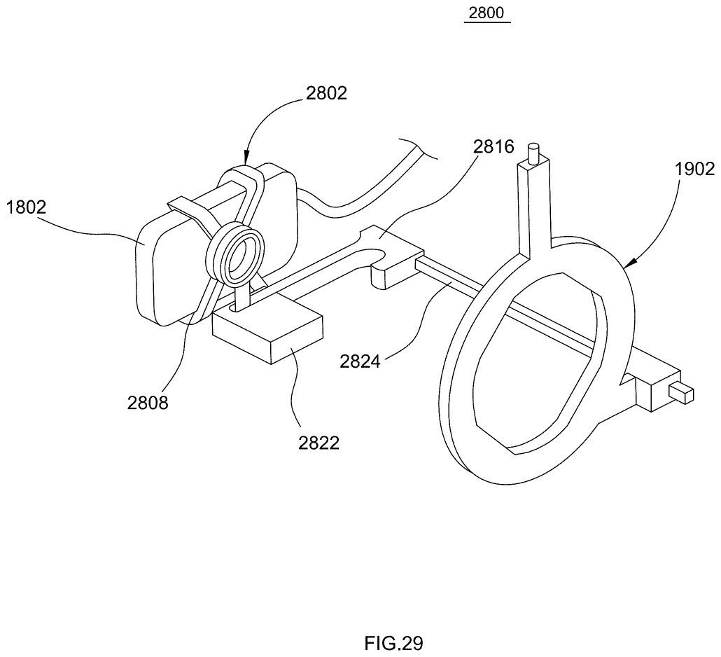

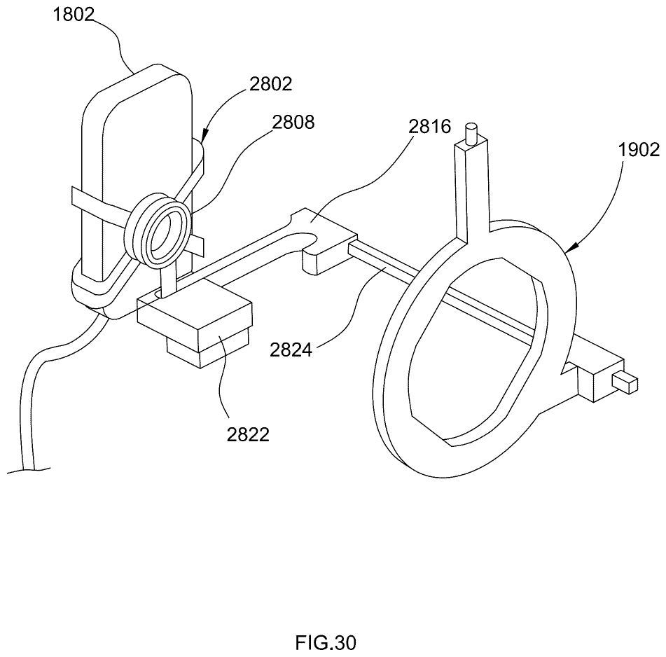

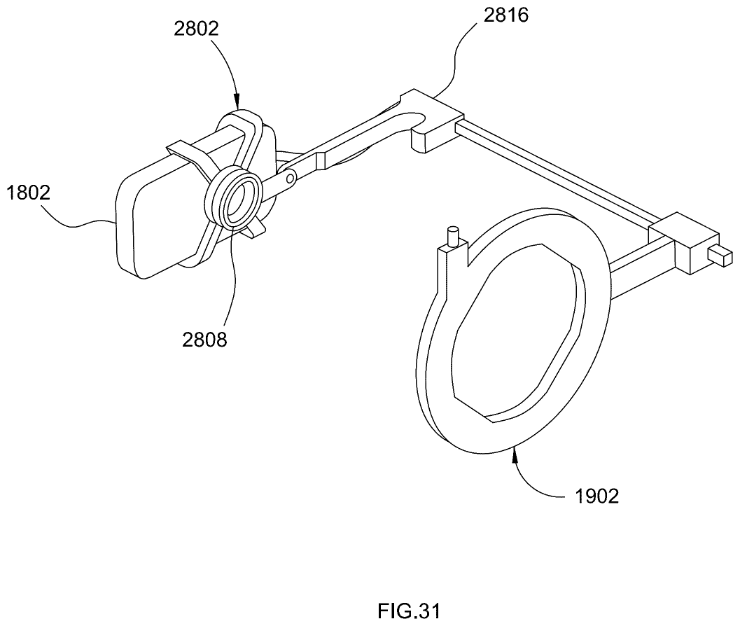

FIG. 28 shows an exploded perspective view of a dental imaging sensor holder system 2800, in accordance with some embodiments. In this system 2800 the sensor holder 2802 operates similarly to that of sensor holder 2100 in that is has opposing grasping portions 2806 that extend from a circular boss 2804. A positioning member 2808 includes a ring portion 2812 that fits over the boss 2804, and has an attachment portion 2810 that extends from the ring portion 2812, and has an attachment post 2814 that extends outward from the attachment portion 2810. The attachment post is configured to fit into a bore 2818 at the end of a holder extension 2816, which connects to the holder arm 2824 at the opposite end of the holder extension 2816. The holder arm 2824 includes a pair of posts 2826 which fit into corresponding bores in the base 2820 of the holder extension 2816. A lock block 2822 is configured to fit over the end of the holder extension 2816 and the attachment portion 2810 to lock them in place. As in previously described embodiments, there is a coupler 2828 the fits on the holder arm 2824 and allows connection of the aiming ring 1902. The coupler includes a square bore 2830 through which the holder arm 2824 can pass, and an attachment bore 2832 into which the attachment port of the legs of the aiming ring 1902 can fit to hold the aiming ring 1902 in place. FIG. 29 shows the system 2800 assembled for an upper or lower imaging position. That is, to capture an image of the upper teeth or the lower teeth. In FIG. 30, the sensor holder 2802 is rotated ninety degrees. The boss 2804, being circular, can rotate in the attachment ring 2812 to reposition the sensor for a different image. In FIG. 31 the system 2800 is configured for a bitewing view where the holder extension 2816 and the attachment portion 2810 are axially aligned. That is, the positioning member 2808 is rotated ninety degrees about the attachment post 2814 in the bore 2818. Note that in FIG. 31 the aiming ring 812 has also be repositioned on the coupler to centrally align the sensor 1802 with the aiming ring 1902.

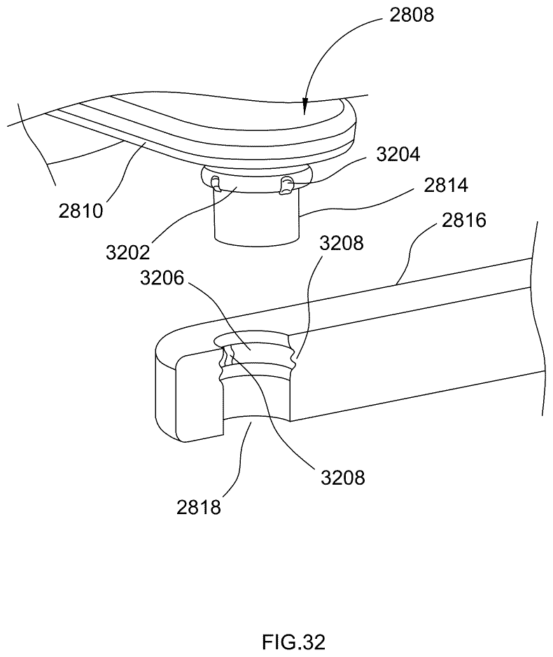

FIG. 32 shows a cutaway detail of a distal end of a holder arm 2816 and the positioning member 2808, in accordance with some embodiments. The attachment post 2814 includes an annular ridge 3202 around the attachment post 2814 that includes a plurality of depressions 3204 that are voids that extend into the annular grove 3202. The bore 2818 through the holder arm 2816 includes an annular groove 3206 that corresponds to the annular ridge 3204, and there are protrusions 3208 that extend inward in the annular groove 3206. When the attachment post 2814 is pressed into the bore 2818, the annular ridge 3202 fits into the annular groove 3206, and when the positioning member 2808 is rotated about the attachment post, at certain positions (as in FIGS. 18-20) the protrusions 3208 align with and fit into the depressions 3204 to create a detent feature. This holds the sensor holder 2802 in various desired positions.

The disclosed multi-configurable dental radiograph sensor holder system eliminates the need for multiple separate sensor holder attachments for each of the various views commonly taken by dental professionals. Using the inventive multi-configurable dental radiograph sensor holder system, a dental professional can simply reconfigure portions of the system by, for example, rotating the sensor holder relative to the positioning ring, articulate the hinge linkage, and adjust the aiming ring coupling for the desired view. Anterior, posterior, and bite wing views can all be taken with the same structure by simply adjusting the positions of the sensor holder, hinge linkage, and aiming ring without having to have an entire kit of various attachments for each of the different views. This greatly simplifies the process of collecting dental images, as well as the process of autoclaving or otherwise sterilizing the components of the radiograph sensor holder.

The claims appended hereto are meant to cover all modifications and changes within the scope and spirit of the present invention.

What is view claimed is:

1. A dental imaging sensor holder comprising:
first and second opposing arms each having a channel configured to receive an edge of a dental imaging sensor;
a base connecting the arms, at least one arm being slidably movable relative to the base to accommodate sensors of different widths; and
at least one connector channel formed on an exterior of at least one of the arms, the connector channel being configured to receive a holder arm in at least two different orientations.

2. The dental imaging sensor holder of claim 1, wherein each connector channel includes at least one retention shoulder extending partially across the channel to retain the holder arm within the channel.

3. The dental imaging sensor holder of claim 1, wherein the base includes a detent mechanism configured to selectively hold the first and second opposing arms at predetermined separations corresponding to widths of standard dental imaging sensors.

4. The dental imaging sensor holder of claim 1, wherein the connector channel extends through a corner region of one of the arms such that the holder arm can be inserted in either of two perpendicular directions.

5. The dental imaging sensor holder of claim 1, wherein the connector channel is square in cross-section and the holder arm is a rod having a square cross-section configured to slidably fit within the connector channel.

6. The dental imaging sensor holder of claim 1, further comprising a plurality of connector channels arranged on different sides of the arms, each connector channel being shaped and sized to interchangeably receive the holder arm.

7. The dental imaging sensor holder of claim 1, wherein the first and second opposing arms each include an interior sensor-receiving channel configured to receive opposite edge portions of the dental imaging sensor.

8. The dental imaging sensor holder of claim 1, wherein the base is rigidly connected to one of the arms and slidably connected to the other arm to permit relative translation between the arms.

9. The dental imaging sensor holder of claim 8, wherein the base includes a guide rail and the slidable arm includes a mating groove that engages the guide rail to constrain motion of the slidable arm.

10. The dental imaging sensor holder of claim 1, wherein the connector channel is open to an exterior surface of the arm to allow insertion of the holder arm laterally into the connector channel.

11. A dental imaging sensor holder comprising:
a pair of opposing grasping portions configured to clasp opposing sides of a dental imaging sensor;
a connector extension pivotably coupled to the grasping portions, the connector extension including an attachment bore; and
a holder arm having an attachment post insertable into the attachment bore in a first orientation or a second orientation perpendicular to the first orientation, wherein the attachment post and the attachment bore each have a square cross-section to prevent relative rotation of the holder arm and the connector extension.

12. The dental imaging sensor holder of claim 11, wherein the connector extension includes both an axial bore and a cross bore, and the attachment post is selectively insertable into either the axial bore or the cross bore.

13. The dental imaging sensor holder of claim 11, wherein the connector extension is rotatable relative to the grasping portions between a plurality of indexed positions.

14. The dental imaging sensor holder of claim 13, wherein the connector extension and the grasping portions include detent features configured to retain the connector extension in at least one of the indexed positions.

15. The dental imaging sensor holder of claim 11, wherein the grasping portions are movable relative to one another to accommodate dental imaging sensors of different sizes.

16. The dental imaging sensor holder of claim 15, wherein the grasping portions include a gear-and-tooth engagement mechanism that constrains separation of the grasping portions.

17. The dental imaging sensor holder of claim 16, further comprising a biasing member urging the grasping portions toward one another.

18. The dental imaging sensor holder of claim 11, wherein the holder arm is a rod having a square cross-section, and the attachment bore of the connector extension is a square bore sized to slidably receive the holder arm.

19. The dental imaging sensor holder of claim 11, further comprising an aiming ring coupler mounted on the holder arm and configured to hold an aiming ring in alignment with the dental imaging sensor.

20. The dental imaging sensor holder of claim 19, wherein the aiming ring coupler is repositionable between a central position and an offset position relative to the holder arm.
